(19) United States
(12) Patent Application Publication
(10) Pub. No.: US 2006/0286039 A1
(43) Pub. Date: Dec. 21, 2006

(54) Novel treatment for pathological aggression

(76) Inventors: James Meyerhoff, Silver Spring, MD (US); Lucille Lumley, Towson, MD (US)

Correspondence Address: Glenna Hendricks, P.O. Box 2509, Fairfax, VA, 22031-2509, US

(21) Appl. No.: 11/512,165

(22) Filed: Aug. 30, 2006

Related U.S. Application Data
(63) Continuation-in-part of application No. 10/357,431, filed on Feb. 4, 2003, now abandoned.

(51) Int. Cl. A61K 38/04; A61K 31/66; A61K 31/19; A61L 9/04

(52) U.S. Cl. 424/45; 514/19; 514/120; 514/574

(57) ABSTRACT

The use of compounds belonging to the class of substituted pentanedioic acids to control aggressive behavior provides new means of treatment for this syndrome.

This application is a continuation-in-part of application Ser. No. 10/357,431 filed Feb. 4, 2003, now pending.

### BACKGROUND AND FIELD OF THE INVENTION

This invention relates to controlling aggressive behavior by using agents to inhibit enzymatic conversion of NAAG to glutamate (Glu).

A common problem in modern society is inappropriate aggression. Although aggressive behavior may be instrumental and adaptive, it may also be maladaptive, pathological or morbid. Pathological aggression is associated with a large range of conditions, as well as brain injuries and a variety of medical illnesses. It would be essential to be able to regulate aggressive behavior independent of management of an underlying medical condition. This is particularly true because disruptive behavior must be resolved promptly, even if the treatment of underlying condition might be long-term. Aggressive, disruptive behavior increases costs and risks to patient (as well as other patients and staff) and also interferes with administration of medical care. Other antipsychotic agents have been used to treat aggression often accompanying schizophrenia and in Alzheimer's Disease (AD), but usefulness of many agents previously used has been limited due to unacceptable extrapyramidal reactions. Newer (“atypical”) antipsychotics have had fewer extrapyramidal reactions, but have shown other, serious, side effects. It has been shown that behavioral problems (e.g., agitation, aggression) can accompany psychosis. However, many agents previously used to control aggressive behavior carry unwanted side effects. As an example, one of the first compounds used to treat aggression in institutional settings was thorazine. The drowsiness, often to the point of stupor, was an unwelcome side effect. Other agents used to treat aggression are effective only against aggression arising from a specific disorder.

It is known that the neurotransmitter, N-Acetyl-aspartyl-glutamate (NAAG), the most abundant neuropeptide in the central nervous system, can be released from neurons in response to depolarization. NAAG then can be enzymatically converted to the excitatory neurotransmitter, glutamate (Glu), by the enzyme alpha-linked acidic dipeptidase (NAALADase), also known as NAAG-hydrolyzing enzyme or glutamate carboxy peptidase II (GCP II [E.C. number 3.4.17.21]). This important reaction occurs under both normal and pathophysiological conditions of the nervous system.

Anxiety and aggression are distinctly different syndromes. Anxious, fearful animals avoid conspecifics and do not attack. Since aggression and fear/anxiety are clearly distinctly different behaviors, are mediated via different mechanisms, and different pharmacological interventions are required to modify these behaviors. A dissociation between anxiety and aggression is described by Neumann (Neumann ID, “Brain mechanisms underlying emotional alterations in the peripartum period in rats.” Depress Anxiety. 2003; 17(3):111-21) in maternal aggression, wherein the lactating female rat demonstrates reduced anxiety and displays increased aggressive behavior. This occurred in rats bred for high as well as low levels of anxiety.

Lack of concordance between aggression and anxiety has also been demonstrated in humans. Koh et al. (Koh K B, Kim C H, Park J K, “Predominance of anger in depressive disorders compared with anxiety disorders and somatoform disorders”, J Clin Psychiatry. 2002, 63(6):486-92) found that patients with depressive disorder had significantly higher levels of anger in comparison with patients with anxiety disorder. On a hostility scale, the depressive disorder group scored significantly higher than the anxiety disorder group. In summary, depressive disorder patients are more likely to have anger than are those with anxiety disorder.

If anxiety were a critical component of aggressive behavior, it would be expected that well-established anxiolytics such as benzodiazedpines would uniformly decrease aggressive behavior. However, it has been found that two different benzodiazedpines, Midazolam and Triazolam, each significantly increases aggressive behavior in rats. In an experimental laboratory paradigm, Ben-Porath and Taylor (Ben-Porath D D, Taylor S P, “The effects of diazepam (valium) and aggressive disposition on human aggression: an experimental investigation”, Addict Behav. 2002; 27(2):167-77) reported that diazepam increased aggressive behavior in normal human volunteers. Finally, Kalachnik et al. (Kalachnik J E, Hanzel T E, Sevenich R, Harder S R, “Brief report: clonazepam behavioral side effects with an individual with mental retardation”, J Autism Dev Disord. 2003; 33(3):349-54) reported that benzodiazepines have been shown to induce aggressive behavior in patients. (This is contra to previously reported findings. However, the newer discoveries in humans in the cited article are deemed particularly probative.)

Long-term individual housing of mice increases aggression, indicated by shorter latency to attack conspecifics; this phenomenon has been termed isolation-induced aggression. Acute administration of antidepressants and anxiolytics, including 5-HT1A receptor agonists, reduce isolation-induced aggression (White et al., 1991; Skolnick et al., 1985). Some NMDA antagonists reduce both isolation-induced aggression (Belozertseva and Bespalov, 1999) and opioid withdrawal induced aggression (Sukhotina and Bespalov, 2000). Since many NMDA antagonists, including ketamine, PCP and MK-801 have psychomimetic effects (see Wedzony et al., 2000), there is interest in developing novel glutamatergic compounds that have therapeutic potential but fewer adverse side effects.

A class of NAALADase inhibitors is disclosed for purposes of treating ischemia in U.S. Pat. No. 5,795,877, which is incorporated herein by reference in its entirety. The pentanedioic acids having a hydroxyphosphinyl moiety at the 2 position of the pentanedioic acids are particularly taught. U.S. Pat. No. 6,228,888, which is incorporated herein in by reference in its entirety, teaches, additionally, other pentanedioic acids having sulfanyl alky substituted benzyl groups at the 2 position of the pentanedioic acid. The agents of that patent are disclosed for use in treating anxiety disorders. There is no suggestion therein that the NAALADase agents have use for amelioration of aggressive activity.

### SUMMARY OF THE INVENTION

This invention relates to the use of compositions containing compounds belonging to the general class of substituted pentanedioic acids, where the substituted moiety at the 2 position of the acid might be (subclass 1) a sulfanyl alkyl group; (subclass 2) an halogenated benzyl and a phosphinyl group or (subclass 3) a phosphonomethyl group, for prevention, management and/or treatment of hyperaggressive behavior arising from environmental or social conditions, injury or disease. Compounds including subclasses 1,2 & 3 as well as [2-(pentafluorophenylmethyl)hydroxyphosphinyl]methyl-pentanedioic acid, and/or other related substituted pentanedioic acids that inhibit NAALADase or mimic NAAG, and/or 2-(phosphonomethyl)-pentanedioic acid (2-PMPA) and/or beta-N-acetyl-aspartyl-glutamate (NAAG) and/or alpha N-acetyl-aspartyl-glutamate are appropriate means for prevention, management and/or treatment of hyperaggressive behavior arising from environmental or social conditions, injury or disease. While these agents have been shown to be useful in treatment of anxiety, their use in treatment of aggression as taught herein had not previously been known. Particularly, compositions containing compounds of subclasses 1, 2 or 3 to include [2-[(pentafluorophenylmethyl)hydroxyphosphinyl]methyl-pentanedioic acid, and/or other related substituted pentanedioic acids that inhibit NAALADase or mimic NAAG, and/or 2-(phosphonomethyl)-pentanedioic acid (2-PMPA) and/or beta-N-acetyl-aspartyl-glutamate (beta-NAAG, a non-hydrolysable analog of the naturally-occurring alpha-NAAG) and/or alpha N-acetyl-aspartyl-glutamate have use as appropriate means for prevention, management and/or treatment of hyperaggressive behavior arising from environmental or social conditions, injury or disease.

### DETAILED DESCRIPTION OF THE INVENTION

Because enzymatic hydrolysis of NAAG form NAA and the more potent excitatory amino acid neurotransmitter, Glu, NAAG has been regarded as a storage form of synaptic Glu. NAALADase has been proposed to affect downstream neuronal excitability by regulating the synaptic availability of Glu. Thus an inhibitor of NAALADase activity would diminish glutamatergic tone by convergent mechanisms. Such mechanisms include directly inhibiting liberation of Glu from the precursor NAAG, resulting in increased levels of NAAG. NAAG itself can diminish overall glutamatergic tone via two mechanisms: (1) NAAG can inhibit Glu release through activation of presynaptic mGluR3 receptors and (2) because NAAG has only a fraction of the excitatory potency of glutamic acid at the NMDA receptor, it can act as a glutamate antagonist at that receptor.

Inhibiting enzymatic conversion of NAAG to glutamate using compounds of subclasses 1, 2 or 3, or 2-PMPA is a useful approach because the convergent triple mechanism may require less drug than would an approach aimed solely at blocking the NMDA receptor. Drugs blocking the NMDA receptor have been associated with unacceptable behavioral side effects. Beta-NAAG has also been identified as useful for treatment of hyperaggressive behavior. However, the mechanism of action is different from that of other active anti-aggression agents identified herein. Beta-NAAG is a non-hydrolyzable analog of NAAG reported to protect cultured neurons against both hypoxia and NMDA-induced injury. The inventors have also found that Beta-NAAG was highly protective in vivo against spinal injury induced by Dynorphin-A. Although it has been suggested that NAAG may be neuroprotective via agonist effects at mGluR3 receptors, it has been reported that beta-NAAG is an antagonist at mGluR3 receptors (Lea et al., 2001). Accordingly, the beneficial effects of beta-NAAG disclosed herein would likely be mediated via reduction of synaptic availability of Glu, or direct or indirect antagonism at the NMDA receptor.

It is well-established that housing in isolation for significant periods of time will enhance the natural tendency of caged resident mice to attack an “intruder” mouse placed in their home cage. The isolation model in mice is the most appropriate model for screening drugs for anti-aggression potential. In order to test whether exposure to an inhibitor of NAALADase of the class identified above would ameliorate aggressive tendencies the NAALADase inhibitor [2-[(pentafluorophenylmethyl)hydroxy-phosphinyl]methy-1-pentanedioic acid (2-PMPA), was administered prior to a social interaction test to determine if such agents would inhibit aggressive behavior in SJL mice that had been individually housed for eight months. [2-[(pentafluoro-phenylmethyl) hydroxyphosphinyl]methyl-pentanedioic acid (30 mg/kg, i.p.) was administered 30 minutes prior to a social interaction test to pharmacologically naive male mice that had been selected and behaviorally shaped to be hyperaggressive, and had consistently shown markedly aggressive behaviors in prior interactions with intruder mice. Administration of [2-[(pentafluorophenylmethyl)hydroxyphosphinyl]methyl-pentanedioic acid (30 mg/kg, i.p.) inhibited aggression, indicated by greater latencies to display tail-rattling, attack and bite, and fewer tail-rattling responses to a non-aggressive conspecific, relative to vehicle. In addition, fewer mice that received [2-[(pentafluorophenylmethyl)hydroxyphosphinyl]methyl-pentanedioic acid (30 mg/kg) initiated attack, relative to those that received vehicle.

It was hypothesized that similar to the effects of NMDA antagonists, NAALADase inhibition and therefore increased NAAG, would inhibit aggressiveness under conditions of high basal levels of aggressiveness. The NAALADase inhibitor [2-[(pentafluorophenylmethyl)hydroxyphosphinyl]methyl-pentanedioic acid was administered prior to a social interaction test in a novel cage and measured behavioral responses to a non-aggressive conspecific; a cage designed to lower the levels of aggressiveness was used.

Principles of laboratory animal care (NIH publication No. 85-23, revised 1985) were followed. Male SJL subject mice (30-35 g; Jackson Laboratories) were housed in reverse 12:12 light-dark cycle (lights off 0900) in a temperature (20-23 .degree. C.) and humidity (50-20%) controlled room, with food and water available ad libitum and were housed individually in large cages (48×27×20 cm) for 8 months, during which time subjects were exposed weekly to non-aggressive C57BL/6 mice that had received olfactory bulbectomies (obx), since these mice do not attack (Denenberg et al., 1973). In addition, these SJL mice had been used to socially defeat mice that were used as subjects in numerous other studies. These highly aggressive SJL had no previous drug exposure. At the end of 8 months of training, subjects weighed 30-37 grams and had attack latencies of less than 30 sec when exposed to an obx mouse+. During the social interaction test, two C57BL/6 male mice (30-33 g; Jackson Laboratories), which were screened for non-aggressiveness and unfamiliar to the subjects, were used as stimulus mice.

Procedure:

[2-[(pentafluorophenylmethyl)hydroxyphosphinyl]methyl-pentanedioic acid, MW 390.2 (3 mg/kg or 30 mg/kg in 50 mM Hepes in 0.9% saline; pH 7.2; Guilford Pharmaceuticals) or vehicle (50 mM Hepes in 0.9% saline; pH 7.2) was administered intraperitoneally (i.p.; 10 ml/kg) 30 min. prior to a 4 min. social interaction test. Subject mice were placed in a novel cage (48.times.27.times.20 cm) along with a non-aggressive male conspecific and tested under low illumination (red lights) during the dark phase of their cycle. Methods for various social interaction tests reviewed in Crawley et al. (2000). Tests were videotaped and behaviors were scored using a computer program (Hindsight, Scott Weiss, UK), by two observers blind to the treatment. Behavioral measures included 1) measures of aggressiveness or threat: attack, bite, chase or follow, grooming conspecific, tail rattling 2) measures of exploratory and locomotor activity: walking, rearing, digging, 3) measures of defensiveness: defensive posture (crouch, upright) and flight.

Statistics:

One-way analysis of variance was performed for each behavioral measure with drug dose as the independent factor. Data were further probed using Dunnet's test. If a behavioral response was not displayed, a maximum latency of 240 seconds was scored.

Results:

[2-[(pentafluorophenylmethyl)hydroxyphosphinyl]methyl-pentanedioic acid (30 mg/kg, i.p.) inhibited aggressiveness in long term individually housed mice, as indicated by increased latencies to display tail-rattling, attack and bite in GPI-treated mice relative to vehicle-treated mice. Mice that received [2-[(pentafluorophenylmethyl)hydroxyphosphinyl]methyl-pentanedioic acid (30 mg/kg) tended to display fewer tail-rattling responses, relative to vehicle-treated mice. There was no effect of [2-[(pentafluorophenylmethyl)hydroxyphosphinyl]methyl-pentanedioic acid on the number of attacks or bites in mice displaying attacks or bites. Only 4 of 9 mice that received 30 mg/kg [2-[(pentafluorophenylmethyl)hydroxyphosphinyl]methyl-pentanedioic acid attacked and 7 of 10 mice that received 3 mg/kg [2-[(pentafluorophenylmethyl)hydroxyphosphinyl]methyl-pentanedioic acid, while all 8 of 8 vehicle-treated mice attacked. Few mice displayed any defensive responses; there was no effect of [2-[(pentafluorophenylmethyl)hydroxyphosphinyl]methyl-pentanedioic acid on defensive responses.

Administration of the NAALADase inhibitor [2-[(pentafluorophenylmethyl)hydroxyphosphinyl]methyl-pentanedioic acid, which increases level of the peptide neurotransmitter NAAG, inhibited aggressiveness in highly aggressive mice that had been individually housed long-term. Mice that received the higher dose of [2-[(pentafluorophenylmethyl)hydroxyphosphinyl]methyl-pentanedioic acid displayed greater latencies to display tail-rattling, considered a component of aggressive behavior and greater latencies to attack and bite the conspecific, relative to vehicle-treated mice. These effects of inhibited aggression are deemed to have been mediated by its effect as a partial agonist on NMDA receptors.

In the current study, the 4 of 9 mice treated with [2-[(pentafluorophenylmethyl)hydroxyphosphinyl]methyl-pentanedioic acid that attacked tended to have more attacks than the vehicle-treated mice, although this effect was not significant. The effect of NAALADase inhibition on aggression may be mediated by decreased glutamate, either through inhibition of glutamate synthesis from NAAG or through NAAG activation of mGluR presynaptic receptors, which inhibits glutamate release. In the current study, basal levels of aggressiveness were extremely high since subjects were individually housed for 8 months and paired briefly with subordinate mice once/week.

Composition Preparation:

A composition containing alpha N-acetyl-aspartyl-glutamate (alpha-NAAG) is prepared by adding 15 mg alpha-NAAG to 10 ml water. Preferred dosage of about 0.01 to 5 mg./kg can be administered parenterally. (It is understood that the smaller the organism, the higher the mg/kg.) The composition is placed in an atomizer and administered as a spray into the nasal passage.

Composition Preparation

A composition of a beta-N-Acetyl-aspartyl-glutamate (beta-NAAG) was prepared in 5% glucose in half-normal saline. Five mg. of beta-NAAG was added to 10 cc normal saline in 5% glucose. The preparation is appropriate for administration parenterally or by mouth or nasal spray.

The active agents of the invention may be administered in the usual pharmaceutically-acceptable carriers, vehicles and/or adjuvants. The term parenteral as used herein includes, but is not limited to, subcutaneous, intravenous, and intramuscular administration. In the methods of the present invention, the compositions containing the aggression inhibiting active agents may be administered parenterally, orally, by inhalation spray, rectally, nasally, buccally, vaginally, topically, or via an implanted reservoir in dosage formulations containing conventional non-toxic pharmaceutically acceptable carrier.

The compounds may also be administered in the form of sterile injectable preparations, for example, as sterile injectable aqueous or oil-based suspensions. These suspensions can be formulated according to techniques known in the art using suitable dispersing or wetting agents and suspending agents. The sterile injectable preparations may be solutions or suspensions in non-toxic parenterally acceptable diluents or solvents, for example, 1,3-butanediol and other agents that enhance solubility. Among the acceptable vehicles and solvents that may be employed are water, Ringer's solution and isotonic sodium chloride solution. In addition, sterile oils are conventionally employed as solvents or suspending mediums. For this purpose, an oil such as a synthetic mono- or, di-glyceride may be employed. Fatty acids such as oleic acid and its glyceride derivatives, including olive oil and castor oil, especially in their polyoxyethylated forms, are useful in the preparation of injectables. These oil solutions or suspensions may also contain long-chain alcohol diluents or dispersants. Oil based carriers are particularly useful for extending time of active effects.

Additionally, the compounds may be administered orally in the form of capsules, tablets, aqueous suspensions or solutions. Tablets may contain carriers such as lactose and cornstarch, and/or lubricating agents such as magnesium stearate. Capsules may contain diluents including lactose and dried cornstarch. Aqueous suspensions may contain emulsifying and suspending agents combined with the active ingredient. The oral dosage forms may further contain sweetening and/or flavoring and/or coloring agents. The compounds may further be administered rectally in the form of suppositories. These compositions can be prepared by mixing the drug with suitable non-irritating excipients that are solid at room temperature, but liquid at rectal temperature such that they will melt in the rectum to release the drug. Such excipients include cocoa butter, beeswax and polyethylene glycols. The NAALADase inhibitors used in the methods of the present invention may be administered by a single dose, multiple discrete doses or continuous infusion. Since the compounds are small, easily diffusible and relatively stable, they are well suited to continuous infusion. Pump means, particularly subcutaneous pump means, are preferred for continuous infusion.

Dose levels on the order of about 0.1 mg to about 10,000 mg of the active ingredient compound are useful in the treatment of the above conditions, with preferred levels being about 0.1 mg to about 1,000 mg. The specific dose level for any particular patient will vary depend upon a variety of factors, including the activity of the specific compound employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful. The considerations for determining the proper dose levels are well known in the art.

In a preferred embodiment, the NAALADase inhibitors are administered in lyophilized form. In this case, 1 to 100 mg of a NAALADase inhibitor may be lyophilized in individual vials, together with a carrier and a buffer, such as mannitol and sodium phosphate. The compound may be reconstituted in the vials with bacteriostatic water before administration.

In treating aggression, the NAALADase inhibitors are preferably administered orally or parenterally at least 1 to 6 times daily, and may follow an initial bolus dose of higher concentration. As previously mentioned, the NAALADase inhibitors used in the methods of the present invention may be administered in combination with one or more other therapeutic agents. Specific dose levels for these other agents will depend upon the drug profile of the agents. For the methods of the present invention, any administration regimen regulating the timing and sequence of drug delivery can be used and repeated as necessary to effect treatment. Such regimen may include pretreatment and/or co-administration with additional therapeutic agents.

What we claim is:

1. A method of treating or preventing hyperaggressive behavior comprising administration of a composition containing an aggression inhibitory effective amount of at least one NAALADase inhibitor chosen from
a 2 substituted pentanedioic acid wherein the substituent at the 2 position is a sulfanyl alkyl group, a halogenated benzyl group, a phosphinyl group or a phosphonomethyl group, beta-N-acetyl-aspartyl-glutamate (beta-NAAG), alpha-N-acetyl-aspartyl-glutamate, (alpha-NAAG), 1-phosphonomethyl pentanedioic acid, and 2 phosphonomethyl pentanedioic acid (2-PMPA) in a pharmaceutically acceptable carrier.

2. The method of claim 1 wherein the hyperaggressive behavior arises from a physical pathological condition.

3. The method of claim 1 wherein the hyperaggressive behavior arises from an environmental imposition.

4. The method of claim 1 wherein the composition contains 0.5% to 6% of alpha-NAAG, beta-NAAG or 2-PMPA.

5. The method of claim 1 wherein said composition is administered parenterally.

6. The method of claim 1 wherein said composition is administered as a spray.
